(19) United States
(12) Patent Application Publication
(10) Pub. No.: US 2025/0354221 A1
(43) Pub. Date: Nov. 20, 2025

(54) METHODS FOR DETECTING GENE LEVEL COPY NUMBER VARIATION IN BRCA1 AND BRCA2

(71) Applicant: Life Technologies Corporation, Carlsbad, CA (US)

(72) Inventors: Charles Scafe, Palo Alto, CA (US); Fiona Hyland, San Mateo, CA (US)

(21) Appl. No.: 19/283,473

(22) Filed: Jul. 29, 2025

Related U.S. Application Data
(63) Continuation of application No. PCT/US2024/013676, filed on Jan. 31, 2024.
(60) Provisional application No. 63/482,317, filed on Jan. 31, 2023.

(51) Int. Cl. C12Q 1/6886

(52) U.S. Cl. CPC C12Q 1/6886; C12Q 2600/156

(57) ABSTRACT

Methods and systems for detecting gene level copy numbers for BRCA1 and BRCA2 genes include amplifying a nucleic acid sample in a presence of a primer pool to produce a plurality of amplicons. The primer pool may include target-specific primers targeting regions of exons of the BRCA1 and BRCA2 genes and sample ID regions. Overlapping amplicons cover the exons of the BRCA1 and BRCA2 genes. Sample ID amplicons are generated for targeted sample ID regions. The amplicons are sequenced to produce sequence reads. The sequence reads are mapped to a reference genome. Determining whole gene copy numbers for the BRCA1 and BRCA2 genes is based on the number of reads per amplicon for the amplicons associated with the exons of the BRCA1 and BRCA2 genes, respectively, and the number of reads per amplicon for the sample ID amplicons associated with the sample ID regions.

### CROSS-REFERENCE TO RELATED APPLICATIONS

This application is a continuation application of International Patent Application no. PCT/US2024/013676, filed Jan. 31, 2024, which claims the benefit of U.S. Provisional Application No. 63/482,317, filed Jan. 31, 2023. The entire contents of the aforementioned applications are incorporated by reference herein.

### FIELD

The present disclosure relates to methods, systems, and computer-readable media for detecting gene level copy number variation in BRCA1 and BRCA2, and, more specifically, in a tumor sample using nucleic acid sequencing data from targeted sequencing panels and next-generation sequencing (NGS) technology.

### BRIEF DESCRIPTION OF THE DRAWINGS

The novel features are set forth with particularity in the appended claims. A better understanding of the features and advantages will be obtained by reference to the following detailed description that sets forth illustrative embodiments and the accompanying drawings of which:

FIG. 1 illustrates an example of using primer pairs to produce amplicons targeting an exon of BRCA1/2.

FIG. 2 illustrates an example of amplicons designed to cover an exon of BRCA1.

FIG. 3 is a block diagram of an exemplary method for detecting gene level copy number variation in BRCA1 and BRCA2, in accordance with an embodiment.

FIG. 4 is a block diagram of an exemplary system for nucleic acid sequencing, in accordance with an embodiment.

### DETAILED DESCRIPTION

The methods described herein enhance the accuracy of whole gene copy number variation in BRCA1 and BRCA2 genes. Previous methods for calling whole gene copy number variants in BRCA1 and BRCA2 genes may not distinguish between deletions and amplifications. Previous methods may call a detected imbalance between the BRCA1 gene and BRCA2 gene as a deletion of a respective gene. The methods described herein use sample ID amplicons as a normal copy number anchor to enable calling the direction of a whole gene change. The present methods can more accurately distinguish between deletions and amplifications in the whole gene copy number variants in BRCA1 and BRCA2 genes. In addition, the present methods can detect whole gene copy number variations in situations where both BRCA1 and BRCA2 genes are affected by copy number variations.

In various embodiments, DNA (deoxyribonucleic acid) may be referred to as a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. In various embodiments, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA.

In various embodiments, a “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” or NGS refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization.

As used herein, the terms “adapter” or “adapter and its complements” and their derivatives, refers to any linear oligonucleotide which can be ligated to a nucleic acid molecule of the disclosure. Optionally, the adapter includes a nucleic acid sequence that is not substantially complementary to the 3′ end or the 5′ end of at least one target sequences within the sample. In some embodiments, the adapter is substantially non-complementary to the 3′ end or the 5′ end of any target sequence present in the sample. In some embodiments, the adapter includes any single stranded or double-stranded linear oligonucleotide that is not substantially complementary to an amplified target sequence. In some embodiments, the adapter is substantially non-complementary to at least one, some or all of the nucleic acid molecules of the sample. In some embodiments, suitable adapter lengths are in the range of about 10-100 nucleotides, about 12-60 nucleotides and about 15-50 nucleotides in length. An adapter can include any combination of nucleotides and/or nucleic acids. In some aspects, the adapter can include one or more cleavable groups at one or more locations. In another aspect, the adapter can include a sequence that is substantially identical, or substantially complementary, to at least a portion of a primer, for example a universal primer. In some embodiments, the adapter can include a barcode or tag to assist with downstream cataloguing, identification or sequencing. In some embodiments, a single-stranded adapter can act as a substrate for amplification when ligated to an amplified target sequence, particularly in the presence of a polymerase and dNTPs under suitable temperature and pH.

As used herein, “DNA barcode” or “DNA tagging sequence” and its derivatives, refers to a unique short (e.g., 6-14 nucleotide) nucleic acid sequence within an adapter that can act as a ‘key’ to distinguish or separate a plurality of amplified target sequences in a sample. For the purposes of this disclosure, a DNA barcode or DNA tagging sequence can be incorporated into the nucleotide sequence of an adapter.

In various embodiments, target nucleic acids generated by the amplification of multiple target-specific sequences from a population of nucleic acid molecules can be sequenced. In some embodiments, the amplification can include hybridizing one or more target-specific primer pairs to the target sequence, extending a first primer of the primer pair, denaturing the extended first primer product from the population of nucleic acid molecules, hybridizing to the extended first primer product the second primer of the primer pair, extending the second primer to form a double stranded product, and digesting the target-specific primer pair away from the double stranded product to generate a plurality of amplified target sequences. In some embodiments, the amplified target sequences can be ligated to one or more adapters. In some embodiments, the adapters can include one or more nucleotide barcodes or tagging sequences. In some embodiments, the amplified target sequences once ligated to an adapter can undergo a nick translation reaction and/or further amplification to generate a library of adapter-ligated amplified target sequences. Exemplary methods of multiplex amplification are described in U.S. Patent Application Publication No. 2012/0295819, published Nov. 22, 2012, incorporated by reference herein in its entirety.

In various embodiments, the method of performing multiplex PCR amplification includes contacting a plurality of target-specific primer pairs having a forward and reverse primer, with a population of target sequences to form a plurality of template/primer duplexes; adding a DNA polymerase and a mixture of dNTPs to the plurality of template/primer duplexes for sufficient time and at sufficient temperature to extend either (or both) the forward or reverse primer in each target-specific primer pair via template-dependent synthesis thereby generating a plurality of extended primer product/template duplexes; denaturing the extended primer product/template duplexes; annealing to the extended primer product the complementary primer from the target-specific primer pair; and extending the annealed primer in the presence of a DNA polymerase and dNTPs to form a plurality of target-specific double-stranded nucleic acid molecules.

In some embodiments, the methods of the disclosure include selectively amplifying target sequences in a sample containing a plurality of nucleic acid molecules and ligating the amplified target sequences to at least one adapter and/or barcode. Adapters and barcodes for use in molecular biology library preparation techniques are well known to those of skill in the art. The definitions of adapters and barcodes as used herein are consistent with the terms used in the art. For example, the use of barcodes allows for the detection and analysis of multiple samples, sources, tissues or populations of nucleic acid molecules per multiplex reaction. A barcoded and amplified target sequence contains a unique nucleic acid sequence, typically a short 6-15 nucleotide sequence, that identifies and distinguishes one amplified nucleic acid molecule from another amplified nucleic acid molecule, even when both nucleic acid molecules minus the barcode contain the same nucleic acid sequence. The use of adapters allows for the amplification of each amplified nucleic acid molecule in a uniformed manner and helps reduce strand bias. Adapters can include universal adapters or propriety adapters both of which can be used downstream to perform one or more distinct functions. For example, amplified target sequences prepared by the methods disclosed herein can be ligated to an adapter that may be used downstream as a platform for clonal amplification. The adapter can function as a template strand for subsequent amplification using a second set of primers and therefore allows universal amplification of the adapter-ligated amplified target sequence. In some embodiments, selective amplification of target nucleic acids to generate a pool of amplicons can further comprise ligating one or more barcodes and/or adapters to an amplified target sequence. The ability to incorporate barcodes enhances sample throughput and allows for analysis of multiple samples or sources of material concurrently.

In this application, “reaction confinement region” generally refers to any region in which a reaction may be confined and includes, for example, a “reaction chamber,” a “well,” and a “microwell” (each of which may be used interchangeably). A reaction confinement region may include a region in which a physical or chemical attribute of a solid substrate can permit the localization of a reaction of interest, and a discrete region of a surface of a substrate that can specifically bind an analyte of interest (such as a discrete region with oligonucleotides or antibodies covalently linked to such surface), for example. Reaction confinement regions may be hollow or have well-defined shapes and volumes, which may be manufactured into a substrate. These latter types of reaction confinement regions are referred to herein as microwells or reaction chambers, and may be fabricated using any suitable microfabrication techniques. Reaction confinement regions may also be substantially flat areas on a substrate without wells, for example.

A plurality of defined spaces or reaction confinement regions may be arranged in an array, and each defined space or reaction confinement regions may be in electrical communication with at least one sensor to allow detection or measurement of one or more detectable or measurable parameter or characteristics. This array is referred to herein as a sensor array. The sensors may convert changes in the presence, concentration, or amounts of reaction by-products (or changes in ionic character of reactants) into an output signal, which may be registered electronically, for example, as a change in a voltage level or a current level which, in turn, may be processed to extract information about a chemical reaction or desired association event, for example, a nucleotide incorporation event. The sensors may include at least one chemically sensitive field effect transistor (“chemFET”) that can be configured to generate at least one output signal related to a property of a chemical reaction or target analyte of interest in proximity thereof. Such properties can include a concentration (or a change in concentration) of a reactant, product or by-product, or a value of a physical property (or a change in such value), such as an ion concentration. An initial measurement or interrogation of a pH for a defined space or reaction confinement regions, for example, may be represented as an electrical signal or a voltage, which may be digitalized (e.g., converted to a digital representation of the electrical signal or the voltage). Any of these measurements and representations may be considered raw data or a raw signal.

As used herein, a “somatic variation” or “somatic mutation” can refer to a variation in genetic sequence that results from a mutation that occurs in a non-germline cell. The variation can be passed on to daughter cells through mitotic division. This can result in a group of cells having a genetic difference from the rest of the cells of an organism. Additionally, as the variation does not occur in a germline cell, the mutation may not be inherited by progeny organisms.

In some embodiments, the targeted sequencing panel comprises the Oncomine BRCA Research NGS Assay available from Thermo Fisher Scientific (SKU A32840 or SKU A32841). The Oncomine BRCA Research NGS Assay covers 100% of all exons of BRCA1/2 with 265 amplicons (targeted regions) using primer pairs. The assay is compatible with DNA samples extracted from FFPE as well as blood samples and with automated and manual library preparation methods. The panel includes eight sample ID (SID) amplicons. The sample ID amplicons are distributed on eight chromosomes unlinked to each other and not on the chromosomes 13 and 17 that contain the BRCA1 and BRCA2 genes.

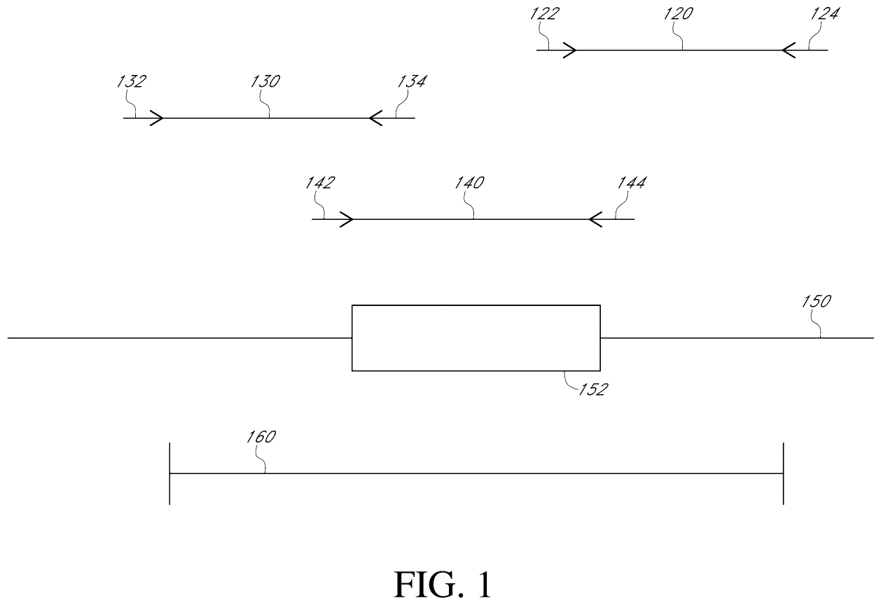

FIG. 1 illustrates an example of using primer pairs to produce amplicons targeting an exon of BRCA1/2. Amplicons 120, 130 and 140 partially overlap each other and together cover an exon 152 of the reference sequence 150, which includes the BRCA1 or BRCA2 gene. The primer pairs 122 and 124 for amplicon 120, primer pairs 132 and 134 for amplicon 130, and primer pairs 142 and 144 for amplicon 140, specifically target regions that overlap the exon 152. The range 160 is an example of the exon coverage region for the cluster of amplicons 120, 130 and 140. Amplification of the target regions of a nucleic acid sample corresponding to target-specific primer pairs 122 and 124, 132 and 134, 142 and 144 can produce multiple copies of amplicons 120, 130 and 140, respectively. Amplification of the amplicons 120, 130 and 140 in the region of the exon 152 would produce a high density of amplicons for the exons of the BRCA1 and BRCA2 genes. The example of a particular arrangement of the amplicons 120, 130 and 140 with respect to exon 152 is for illustrative purposes only and is not limiting.

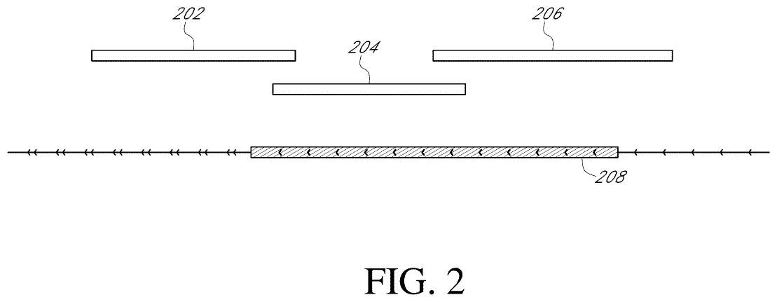

FIG. 2 illustrates an example of amplicons designed to cover an exon of BRCA1. In this example, three amplicons 202, 204 and 206 are designed to span a region that includes an exon 208 of a BRCA1 reference sequence. This example is for illustrative purposes and is not limiting.

In some embodiments, a group of amplicons may cover an exon and regions adjacent to the exon. The number of amplicons in a group of amplicons may range from two to over 50. Typical numbers of amplicons in a group covering an exon is three to five. One or more amplicons in a group may not overlap the exon, however any one amplicon overlaps at least one other amplicon in the group. The group of amplicons together may cover the exon and regions adjacent to the exon.

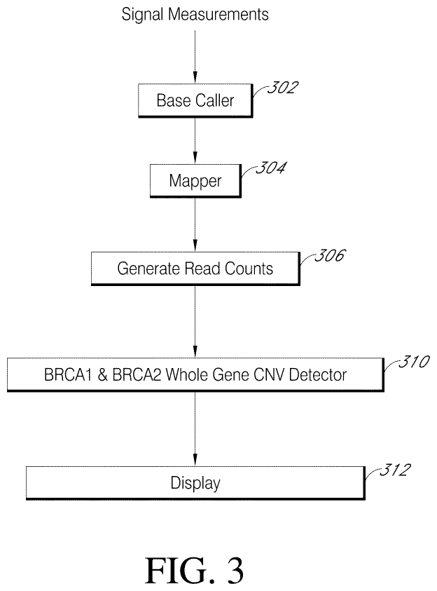

FIG. 3 is a block diagram of an exemplary method for detecting gene level copy number variation in BRCA1 and BRCA2, in accordance with an embodiment. Signal measurements may be provided to a processor by a nucleic acid sequencing device. In some embodiments, each signal measurement represents a signal amplitude or intensity measured in response to an incorporation or non-incorporation of a flowed nucleotide by sample nucleic acids in microwells of a sensor array. For an incorporation event, the signal amplitudes depend on the number of bases incorporated at one flow. For homopolymers, the signal amplitudes increase with increasing homopolymer length. The processor may apply a base caller 302 to generate base calls for a sequence read by analyzing flow space signal measurements. The signal measurements may be raw acquisition data or data having been processed, such as, e.g., by scaling, background filtering, normalization, correction for signal decay, and/or correction for phase errors or effects, etc. The base calls may be made by analyzing any suitable signal characteristics (e.g., signal amplitude or intensity). The structure and/or design of a sensor array, signal processing and base calling for use with the present teachings may include one or more features described in U.S. Pat. Appl. Publ. No. 2013/0090860, Apr. 11, 2013, incorporated by reference herein in its entirety.

Once the base sequence for the sequence read is determined, the sequence reads may be provided to mapper 304. The mapper 304 aligns the sequence reads to a reference genome to determine aligned sequence reads and associated mapping quality parameters. The sample may include a sample ID amplicons associated with different chromosomes than the BRCA1 (chromosome 17) and BRCA2 (chromosome 13). The base caller 302 and mapper 304 may process the sample ID amplicons along with the amplicons associated with the exons of BRCA1 and BRCA2. Methods for aligning sequence reads for use with the present teachings may include one or more features described in U.S. Pat. Appl. Publ. No. 2012/0197623, published Aug. 2, 2012, incorporated by reference herein in its entirety. The aligned sequence reads may be provided for further processing, for example, in a BAM file.

The aligned sequence reads are associated with amplicons at specific locations relative to the reference genome. The read counts block 306 determines the number of reads per amplicon, referred to as coverage. The read counts block 306 determines the number of reads per amplicon for amplicons targeting the exons of the BRCA1 and BRCA2 genes and the sample ID amplicons.

The whole gene CNV detector 310 may apply the following steps:
a. Divide the number of reads per amplicon for amplicons associated with exons of the BRCA1 gene by the total number of reads in the sample to form normalized read counts per amplicon associated with the BRCA1 gene.
b. Calculate the mean and standard deviation of the normalized read counts per amplicon associated with the BRCA1 gene.
c. Divide the number of reads per amplicon for amplicons associated with exons of the BRCA2 gene by the total number of reads in the sample to form normalized read counts per amplicon associated with the BRCA2 gene.
d. Calculate the mean and standard deviation of the normalized read counts per amplicon associated with the BRCA2 gene.
e. Apply a t-test based on the means and standard deviations calculated for the BRCA1 and BRCA2 genes to determine a test statistic and associated p-value.
f. Compare the p-value to a first threshold to detect a whole gene deletion or amplification if the p-value is less than the first threshold. An exemplary value for first threshold is given in Table 1, item a, “P-value cutoff for Gene1<Gene2.”
g. Calculate the PHRED score based on the p-value,

$$PHRED score = - 10 log ( p - value )$$

For example, a PHRED score of 40 corresponds to a p-value threshold of 10⁻⁴.
h. Compare each gene's (standard deviation)/mean (coefficient of variation, “CV”) to a second threshold, or maximum CV, and call a whole gene CNV if the (standard deviation)/mean is less than the second threshold. An exemplary value for the second threshold is given in Table 1, item d, “Maximum CV.”
i. Divide the number of sample ID reads by the total number of reads in the sample to form normalized read counts per amplicon of the sample ID amplicons.
j. Compare the mean of the normalized read counts per amplicon associated with BRCA1 to the mean of the normalized read counts per amplicon of the SID amplicons. Compare the mean of the normalized read counts for BRCA2 to the mean of the normalized read counts per amplicon of the SID amplicons. Table 2 includes the decision logic for comparisons of the respective means of normalized read counts per amplicon for BRCA1, BRCA2 and SID amplicons.

The coefficient of variation (CV) may be calculated based on the ratio of the mean of the normalized read counts to the standard deviation of the normalized read counts. The CV may be calculated, respectively, for the BRCA1 gene, the BRCA2 gene, and the sample ID amplicons based on the respective normalized read counts. Table A gives examples the CVs for BRCA1 (“BRCA1CVs”), BRCA2 (“BRC21CVs”), and sample ID (“sampleIDCVs”) implemented in R programming code, where “sd” refers to the respective standard deviation and “colMeans” refers to the respective means.

TABLE A

| | |
|---|---|
| 1 | RatioNormCV<-data.frame(row.names=c(“BRCA1”, “BRCA2”, “SampleID”)) |
| 2 | BRCA1CVs<-apply(RatioNorm[grep(“BRCA1”, Ratio.sorted$Pool),],2, |
| 3 | sd)/colMeans(RatioNorm[grep(“BRCA1”, Ratio.sorted$Pool),]) |
| 4 | BRCA2CVs<-apply(RatioNorm[grep(“BRCA2”, Ratio.sorted$Pool),],2, |
| | sd)/colMeans(RatioNorm[grep(“BRCA2”, Ratio.sorted$Pool),]) |
| 5 | sampleIDCVs<-apply(RatioNorm[grep(“sid”, Ratio.sorted$Pool),],2, |
| 6 | sd)/colMeans(RatioNorm[grep(“sid”, Ratio.sorted$Pool),]) |
| 7 | |

If BRCA1 and BRCA2 have the same normalized read counts, as determined by the t-test, then each normalized read count is compared to the SID to determine whether there is an amplification, deletion or normal.

The whole gene CNV detector 310 may provide the copy number calls for the BRCA1 and BRCA2 genes and related information in an output file for display 312 to the user.

TABLE 1
Parameters:

| | |
|---|---|
| a. | P-value cutoff for Gene1 < Gene2 single sided. Default value 0.0001. |
| | Name: cnv.brca.gene.threshold (Range: 0 to 1) |
| b. | Minimum distance between two genes to call a GeneCNV. Default value |
| | 1.3. Name: cnv.brca.gene.min.factor (Range 1 to 10) |
| c. | Maximum MAPD for calling a GeneCNV. Name: cnv.brca.gene.max.mapd |
| | (Range 0.05 to 1.0) |
| | i. Default value 0.65 |
| d. | Maximum CV for affected gene for calling a GeneCNV instead of a Large |
| | Gene Rearrangement. Default value 0.41. Name: cnv.brca.gene.max.cv |
| | (Range 0.05 to 1.0) |
| e. | Pvalue cutoff for sample id < GeneX. Default value 0.007. Name: |
| | cnv.brca.gene.sid.gain.threshold (Range 0 to 1) |
| f. | Pvalue cutoff for GeneX < sample id. Default value 0.03. Name: |
| | cnv.brca.gene.sid.loss.threshold (Range 0 to 1) |
| g. | Minimum distance between sid and duplicated gene. Default value 1.4. |
| | Name: cnv.brca.gene.sid.gain.factor (Range 1 to 10) |
| h. | Minimum distance between sid and deleted gene. Default value 1.2. |
| | Name: cnv.brca.gene.sid.loss.factor (Range 1 to 10) |
| i. | Maximum CV for sid for making any direction call. Default value 0.40. |
| | Name: cnv.brca.gene.sid.max.cv (Range 0.05 to 1.0) |

TABLE 2
If BRCA1 < BRCA2:

| | |
|---|---|
| • | CaseI: BRCA1Del Only: if BRCA1<sid: |

| | |
|---|---|
| • | If BRCA2 not <sid and BRCA2 not >sid |

| | |
|---|---|
| • | if BRCA1 CV < threshold and sid CV < threshold |

| | |
|---|---|
| • | BRCA1DEL |

| | |
|---|---|
| • | CaseII: BRCA1Del and BRCA2Dup: if BRCA1<sid and BRCA2 > sid: |

| | |
|---|---|
| • | If BRCA1 CV < threshold and sid CV < threshold and BRCA2 CV < |
| | threshold |

| | |
|---|---|
| • | BRCA1DEL AND BRCA2DUP |

| | |
|---|---|
| • | CaseIII: BRCA1 normal, BRCA2Dup: if BRCA2> sid AND BRCA1 not < sid: |

| | |
|---|---|
| • | If BRCA2 CV < threshold and sid CV < threshold: |

| | |
|---|---|
| • | BRCA2DUP |

| | |
|---|---|
| • | CaseIV: SID below both BRCA1 and 2, BRCA1 and 2 both duplicated to different |
| | extents: |

| | |
|---|---|
| • | If sid < BRCA1 AND sid < BRCA2: |

| | |
|---|---|
| • | If BRCA1 CV < threshold AND BRCA2 CV < threshold AND sid |
| | CV < threshold: |

| | |
|---|---|
| • | BRCA1DUP, BRCA2DUP. |

If BRCA2 < BRCA1:

| | |
|---|---|
| • | Case V: BRCA2Del and BRCA1Dup |

| | |
|---|---|
| • | If BRCA2 < sid AND BRCA1 > sid: |

| | |
|---|---|
| • | If BRCA2 CV < threshold and sid CV < threshold and BRCA1 CV |
| | < threshold: |

| | |
|---|---|
| • | BRCA2DEL, BRCA1DUP. |

| | |
|---|---|
| • | CaseVI: SID below both BRCA1 and 2, BRCA1 and 2 both duplicated to different |
| | extents: |

| | |
|---|---|
| • | If sid < BRCA2 and sid < BRCA1: |

| | |
|---|---|
| • | If BRCA2 CV < threshold AND BRCA1 CV < threshold AND sid |
| | CV < threshold: |

| | |
|---|---|
| • | BRCA2DUP, BRCA1DUP |

| | |
|---|---|
| • | CaseVII: BRCA2 Del, Normal BRCA1: if BRCA2 < sid AND BRCA1 not < sid |
| | AND BRCA1 not > sid: |

| | |
|---|---|
| • | If BRCA2 CV < threshold and sid CV < threshold: |

| | |
|---|---|
| • | BRCA2DEL |

| | |
|---|---|
| • | CaseVIII: BRCA1Dup, Normal BRCA2: if BRCA1 > sid AND BRCA2 not < sid |
| | AND BRCA2 not > sid: |

| | |
|---|---|
| • | If BRCA1 CV < threshold and sid CV < threshold: |

| | |
|---|---|
| • | BRCA1DUP |

If BRCA1 not < BRCA2 AND BRCA1 not > BRCA2:

| | |
|---|---|
| • | CaseIX: Both genes duplicated to same extent: if BRCA1 > sid AND BRCA2 > sid: |

| | |
|---|---|
| • | If BRCA1CV < threshold AND BRCA2 CV < threshold AND sid CV < |
| | threshold: |

| | |
|---|---|
| • | BRCA1DUP, BRCA2DUP |

| | |
|---|---|
| • | CaseX: BRCA1 NOCALL, BRCA2DUP: if BRCA2 > sid AND BRCA1 not > sid: |

| | |
|---|---|
| • | If BRCA2 CV < threshold AND sid CV < threshold: |

| | |
|---|---|
| • | BRCA1NC, BRCA2DUP |

| | |
|---|---|
| • | CaseXI: BRCA1DUP, BRCA2 NOCALL: if BRCA1 > sid AND BRCA2 not > sid: |

| | |
|---|---|
| • | If BRCA1 CV < threshold AND sid CV < threshold: |

| | |
|---|---|
| • | BRCA1DUP, BRCA2NC |

In Table 2, the abbreviations are as follows: mean of the normalized read counts per amplicon for the BRCA1 gene—“BRCA1”, mean of the normalized read counts per amplicon for the BRCA2 gene—“BRCA2”, mean of the normalized read counts per amplicon for the sample ID amplicons—“sid”, deletion—“Del” or “DEL”, duplication—“Dup” or “DUP”, sample ID—“sid”, coefficient of variation (standard deviation divided by mean)—CV, NOCALL—“NC”.

Table B gives an example of an implementation in R programming code of Table 2's step “If BRCA1<BRCA2.” A t-test is applied based on the means and standard deviations of the normalized read counts per amplicon of the BRCA1 and BRCA2 genes, respectively, and the p-value is compared to a p-value threshold (“pvalue.THRESH”). Exemplary values of the p-value threshold, also referred to herein as “first threshold,” is given in Table 1, item a, “P-value cutoff for Gene1<Gene2.” The product of a factor (“min.FACTOR”) times the mean for BRCA1 (“brca1.mean”) is compared to the mean for BRCA2 (“brca2.mean”). Exemplary values for the factor (“min.FACTOR”) are given in Table 1, item b, “Minimum distance between two genes to call a GeneCNV.” In this example, the BRCA1 mean is less than the BRCA2 mean.

TABLE B

| | |
|---|---|
| 1 | if ((BRCA1del_ttest$p.value < pvalue.THRES )&&(min.FACTOR*brca1.mean < |
| 2 | brca2.mean)){ |
| 3 | #BRCA1 < BRCA2 |
| | print(“BRCA1 It BRCA2 by factor of at least wgDelFactor”) |

Table C gives an example of an implementation in R programming code of the logic for Table 2's “Case I: BRCA1 deletion only.” For Table 2's step, “if BRCA1<sid”, a t-test based on the mean and standard deviation of the normalized read counts per amplicon for BRCA1 and the mean and standard deviation of the normalized read counts per amplicon for the SID amplicons, and the p-value is compared to a SID loss p-value threshold (“sid.loss.THRESH”). Exemplary values for the SID loss p-value threshold (“sid.loss.THRESH”) are given in Table 1, item f, “Pvalue cutoff for GeneX<sample id.” A product of a weight (“sid.loss.FACTOR”) times the BRCA1 mean (“brca1.mean”) is compared to the SID mean (“sid.mean”). Exemplary values for the weight (“sid.loss.FACTOR”) are given in Table 1, item h, “Minimum distance between sid and deleted gene.” In this example, the BRCA1 mean is less than the SID mean.

TABLE C

| | |
|---|---|
| 1 | #Casel: BRCA1Del Only. a. test BRCA1<sid by pvalue and factor. b. test BRCA2 not |
| 2 | <sid, not >sid by pvalue or factor. |
| 3 | if ((SID_gtBRCA1_ttest$p.value < sid.loss.THRES) && |
| 4 | (sid.loss.FACTOR*brca1.mean < sid.mean)) { |

Table D gives an example of an implementation in R programming code of the logic for Table 2's step “If BRCA2 not<sid and BRCA2 not>sid.” A t-test is applied based on the mean and standard deviation of the normalized read counts per amplicon for the SID amplicons and the mean and standard deviation of the normalized read counts per amplicon for the BRCA2 gene and the p-value is compared to a SID gain p-value threshold (“sid.gain.THRES”). Exemplary values for the SID gain p-value threshold (“sid.gain.THRES”) are given in Table 1, item e, “Pvalue cutoff for sample id<GeneX.” A product of a weight (“sid.gain.FACTOR”) times the SID mean (sid.mean) is compared to the BRCA2 mean (“brca2.mean”). Exemplary values for the weight (“sid.gain.FACTOR”) are given in Table 1, item g, “Minimum distance between sid and duplicated gene.” A t-test is applied based on the mean and standard deviation of the normalized read counts per amplicon for the SID amplicons and the mean and standard deviation of the normalized read counts per amplicon for the BRCA2 gene and the p-value is compared to a SID loss p-value threshold (sid.loss.THRES). Exemplary values for the SID loss p-value threshold (“sid.loss.THRES”) are given in Table 1, item f, “Pvalue cutoff for GeneX<sample id.” A product of a weight (“sid.loss.FACTOR”) times the BRCA2 mean (“brca2.mean”) is compared to the SID mean (“sid.mean”). Exemplary values for the weight (“sid.loss.FACTOR”) are given in Table 1, item h, “Minimum distance between sid and deleted gene.” For the example of Case I, BRCA2 mean is not less than the sid mean and BRCA2 mean is not greater than the sid mean.

TABLE D

| | |
|---|---|
| 1 | if (((SID_ItBRCA2_ttest$p.value >= sid.gain.THRES) \| \| (sid.gain.FACTOR*sid.mean >= |
| 2 | brca2.mean)) && ((SID_gtBRCA2_ttest$p.value >= sid.loss.THRES) \| \| |
| 3 | (sid.loss.factor*brca2.mean >= sid.mean))) { |

Table E gives an example of an implementation in R programming code of the logic for Table 2's step “if BRCA1 CV<threshold and sid CV<threshold.” The coefficient of variation (CV) for BRCA1 (“BRCA1CVs”) is compared with a maximum CV threshold (“max.CV”). Exemplary values for a maximum CV threshold (“max.CV”), also referred to herein as a second threshold, are given in Table 1, item d, “Maximum CV for affected gene for calling a GeneCNV instead of a Large Gene Rearrangement.” The CV for the sample ID amplicons (“sampleIDCVs”) is compared with a maximum SID CV threshold (“sid.max.CV”). Exemplary values for a maximum SID CV threshold (“sid.max.CV”) are given in Table 1, item i, “Maximum CV for sid for making any direction call.” If the respective CVs are greater than the respective CV maximum thresholds, then the variation is likely due to a large rearrangement in the BRCA1 gene. If the respective CVs are not greater than the respective CV maximum thresholds, then the variation is likely a whole gene deletion of the BRCA1 gene. For the example of Case I in Table 2, the respective CVs are not greater than the respective CV maximum thresholds, which indicates a whole gene BRCA1 deletion, “BRCA1DEL.”

TABLE E

| | |
|---|---|
| 1 | if ((RatioNormCVs[,sample][“BRCA1CVs”][[1]]>max.CV) && |
| 2 | (RatioNormCVs[,sample][“sampleIDCVs”]>sid.max.CV)) { |
| 3 | print(paste0(sample, “ likely has large deletion(s) of BRCA1”)) #If |
| 4 | variation is large, likely large deletion in the gene |
| 5 | wglgDelMatrix[1,sample]<-paste0(“LGD=”, SID_gtBRCA1_Phred) |
| 6 | } |
| 7 | else{ |
| 8 | print(paste0(sample, “ likely has whole gene deletion of BRCA1”)) #If |
| 9 | variation is small, likely whole gene deletion |
| 10 | wglgDelMatrix[1,sample]<-paste0(“WGD=”,SID_gtBRCA1_Phred) |

A median absolute pairwise difference (MAPD) can be calculated for the ratios of the normalized read counts per amplicon and the baseline coverage of adjacent amplicons. Since adjacent amplicons should ideally have the same copy number, finding the median value of the absolute values of the differences of the copy number levels provides an indication of quality. Exemplary values for a threshold for MAPD are given in Table 1, item c, “Maximum MAPD for calling a GeneCNV.” The MAPD values for the sample may provide a quality value for the candidate copy number. Methods for determining MAPD values for copy number variation for use with the present teachings may include one or more features described in U.S. Pat. Appl. Publ. No. 2014/0256571, published Sep. 11, 2014, which is incorporated by reference herein in its entirety.

Table 3 gives results of the present method for whole gene detection for BRCA1 and BRCA2 compared to the previous method. The previous method detected BRCA1/2 gene deletions by comparing the normalized read counts for BRCA1 and BRCA2 to each other. The lowest copy number gene was considered a deletion variant. The present method applies comparisons of the respective normalized read counts for the BRCA1 and BRCA2 to the normalized read counts for the sample ID amplicons. In most cases, the distribution of the sample ID amplicons should reflect the normal gene copy number in the sample. The comparisons of the normalized read counts for each of BRCA1 and BRCA2 individually to the normalized read counts for the sample ID amplicons enables the present method to discriminate between amplification and deletion of the gene.

Tests to determine if the gene level is normal, deleted or amplified were conducted on 16 DNA samples from FFPE tumor samples in wet lab testing and 6 samples in in-silico testing. The inputs for in-silico testing were sequence BAM files from known truth samples. The results were compared with a truth set generated by orthogonal testing. The orthogonal test results were generated by an Oncoscan array copy number assay. Table 3 compares the specificity results across the samples tested using the present method and the previous method. Specificity is defined as TP/(TP+FN)×100, where TP is true positive and FN is false negative. The performance results show high specificity of 100 for the present method for both germline and somatic samples for detection of whole gene CNV for BRCA1 and BRCA2. The performance of the present method shows an improvement over the previous method's specificity of 71.4 for somatic and 80 for germline for detection of whole gene CNV for BRCA1 and BRCA2.

TABLE 3

| | PRESENT | PREVIOUS | PRESENT | PREVIOUS |
|---|---|---|---|---|
| | METHOD | METHOD | METHOD | METHOD |
| WORKFLOW | SOMATIC | SOMATIC | GERMLINE | GERMLINE |
| SPECIFICITY | 100 | 71.4 | 100 | 80 |
| GeneCNV | | | | |

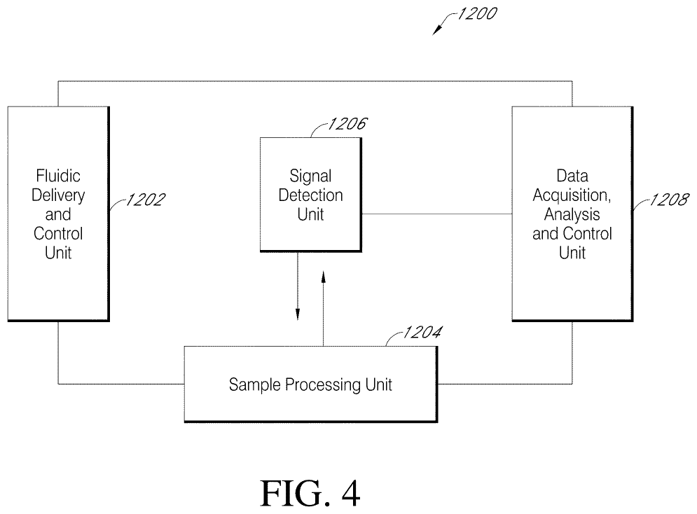

Various embodiments of nucleic acid sequencing platforms, such as a nucleic acid sequencer, can include components as displayed in the block diagram of FIG. 4. According to various embodiments, sequencing instrument 1200 can include a fluidic delivery and control unit 1202, a sample processing unit 1204, a signal detection unit 1206, and a data acquisition, analysis and control unit 1208. Various embodiments of instrumentation, reagents, libraries and methods used for next generation sequencing are described in U.S. Patent Application Publication No. 2009/0127589 and No. 2009/0026082, each of which is incorporated by reference herein in its entirety. Various embodiments of instrument 1200 can provide for automated sequencing that can be used to gather sequence information from a plurality of sequences in parallel, such as substantially simultaneously.

In various embodiments, the fluidics delivery and control unit 1202 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir.

In various embodiments, the sample processing unit 1204 can include a sample chamber, such as flow cell, a substrate, a micro-array, a multi-well tray, or the like. The sample processing unit 1204 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 1206 can include an imaging or detection sensor. For example, the imaging or detection sensor can include a CCD, a CMOS, an ion or chemical sensor, such as an ion sensitive layer overlying a CMOS or FET, a current or voltage detector, or the like. The signal detection unit 1206 can include an excitation system to cause a probe, such as a fluorescent dye, to emit a signal. The excitation system can include an illumination source, such as arc lamp, a laser, a light emitting diode (LED), or the like. In particular embodiments, the signal detection unit 1206 can include optics for the transmission of light from an illumination source to the sample or from the sample to the imaging or detection sensor. Alternatively, the signal detection unit 1206 may provide for electronic or non-photon based methods for detection and consequently not include an illumination source. In various embodiments, electronic-based signal detection may occur when a detectable signal or species is produced during a sequencing reaction. For example, a signal can be produced by the interaction of a released byproduct or moiety, such as a released ion, such as a hydrogen ion, interacting with an ion or chemical sensitive layer. In other embodiments a detectable signal may arise as a result of an enzymatic cascade such as used in pyrosequencing (see, for example, U.S. Patent Application Publication No. 2009/0325145) where pyrophosphate is generated through base incorporation by a polymerase which further reacts with ATP sulfurylase to generate ATP in the presence of adenosine 5′ phosphosulfate wherein the ATP generated may be consumed in a luciferase mediated reaction to generate a chemiluminescent signal. In another example, changes in an electrical current can be detected as a nucleic acid passes through a nanopore without the need for an illumination source.

In various embodiments, a data acquisition analysis and control unit 1208 can monitor various system parameters. The system parameters can include temperature of various portions of instrument 1200, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 1200 can be used to practice variety of sequencing methods including ligation-based methods, sequencing by synthesis, single molecule methods, nanopore sequencing, and other sequencing techniques.

In various embodiments, the sequencing instrument 1200 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 1200 can obtain the sequence information from a single nucleic acid molecule or from a group of substantially identical nucleic acid molecules.

In various embodiments, sequencing instrument 1200 can output nucleic acid sequencing read data in a variety of different output data file types/formats, including, but not limited to: *. fasta, *.csfasta, *seq.txt, *qseq.txt, *.fastq, *.sff, *prb.txt, *.sms, *srs and/or *.qv.

According to various exemplary embodiments, one or more features of any one or more of the above-discussed teachings and/or exemplary embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various exemplary embodiments, one or more features of any one or more of the above-discussed teachings and/or exemplary embodiments may be performed or implemented using appropriately configured and/or programmed non-transitory machine-readable medium or article that may store an instruction or a set of instructions that, if executed by a machine, may cause the machine to perform a method and/or operations in accordance with the exemplary embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, Flash memory, hard drive, tape, CDROM, etc.). Moreover, memory can incorporate electronic, magnetic, optical, and/or other types of storage media. Memory can have a distributed architecture where various components are situated remote from one another, but are still accessed by the processor. The instructions may include any suitable type of code, such as source code, compiled code, interpreted code, executable code, static code, dynamic code, encrypted code, etc., implemented using any suitable high-level, low-level, object-oriented, visual, compiled and/or interpreted programming language.

According to various exemplary embodiments, one or more features of any one or more of the above-discussed teachings and/or exemplary embodiments may be performed or implemented at least partly using a distributed, clustered, remote, or cloud computing resource.

According to various exemplary embodiments, one or more features of any one or more of the above-discussed teachings and/or exemplary embodiments may be performed or implemented using a source program, executable program (object code), script, or any other entity comprising a set of instructions to be performed. When a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, R, Python, Pascal, Basic, Fortran, Cobol, Perl, Java, and Ada.

According to various exemplary embodiments, one or more of the above-discussed exemplary embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such exemplary embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

### EXAMPLES

Example 1 is a method for detecting gene level copy numbers for BRCA1 and BRCA2 genes, including: amplifying a nucleic acid sample in a presence of a primer pool to produce a plurality of amplicons, the primer pool including a plurality of target-specific primers targeting regions of exons of the BRCA1 and BRCA2 genes and a plurality of sample ID regions, wherein the target-specific primers targeting the regions of exons produce overlapping amplicons that cover the exons of the BRCA1 and BRCA2 genes and the target-specific primers targeting the plurality of sample ID regions produce a plurality of sample ID amplicons; sequencing the amplicons to generate a plurality of sequence reads; mapping the sequence reads to a reference genome, wherein the reference genome includes the BRCA1 and BRCA2 genes and the sample ID regions; determining a number of reads per amplicon for the amplicons associated with the exons of the BRCA1 gene, a number of reads per amplicon for the amplicons associated with the exons of the BRCA2 gene and a number of reads per amplicon for the sample ID amplicons associated with the sample ID regions; and determining whole gene copy numbers for the BRCA1 and BRCA2 genes based on the number of reads per amplicon for the amplicons associated with the exons of the BRCA1 gene, the number of reads per amplicon for the amplicons associated with the exons of the BRCA2 gene, and the number of reads per amplicon for the sample ID amplicons associated with the sample ID regions.

Example 2 includes the subject matter of Example 1, and further specifies that the sample ID amplicons are associated with different chromosomes than chromosome 17 containing the BRCA1 gene and chromosome 13 containing the BRCA2 gene.

Example 3 includes the subject matter of Example 1, and further specifies that the sample ID amplicons are distributed on different chromosomes unlinked to each other.

Example 4 includes the subject matter of Example 1, and further specifies that the step of determining whole gene copy numbers further comprises: dividing the number of reads per amplicon for the amplicons associated with the exons of the BRCA1 gene by a total number of reads for the sample to form normalized read counts per amplicon for the BRCA1 gene; and dividing the number of reads per amplicon for the amplicons associated with the exons of the BRCA2 gene by the total number of reads for the sample to form normalized read counts per amplicon for the BRCA2 gene.

Example 5 includes the subject matter of Example 4, and further specifies that the step of determining whole gene copy numbers further comprises: calculating a first mean and a first standard deviation of the normalized read counts per amplicon of the BRCA1 gene; and calculating a second mean and a second standard deviation of the normalized read counts per amplicon of the BRCA2 gene.

Example 6 includes the subject matter of Example 5, and further specifies that the step of determining whole gene copy numbers further comprises applying a t-test based on the first and second means and the first and second standard deviations to determine a p-value.

Example 7 includes the subject matter of Example 6, and further specifies that the step of determining whole gene copy numbers further comprises comparing the p-value to a first threshold.

Example 8 includes the subject matter of Example 7, and further specifies that the step of determining whole gene copy numbers further comprises identifying a whole gene deletion or a whole gene amplification if the p-value is less than the first threshold.

Example 9 includes the subject matter of Example 6, and further specifies that the step of determining whole gene copy numbers further comprises calculating a PHRED score based on the p-value.

Example 10 includes the subject matter of Example 9, and further specifies that the PHRED score is calculated by multiplying a logarithm of the p-value by (−10).

Example 11 includes the subject matter of Example 5, and further specifies that the step of determining whole gene copy numbers further comprises: calculating a first coefficient of variation (CV) by dividing the first standard deviation by the first mean; and calculating a second coefficient of variation (CV) by dividing the second standard deviation by the second mean.

Example 12 includes the subject matter of Example 11, and further specifies that the step of determining whole gene copy numbers further comprises comparing the first coefficient of variation with a second threshold and calling a whole gene copy number variation for the BRCA1 gene if the first coefficient of variation is less than the second threshold.

Example 13 includes the subject matter of Example 11, and further specifies that the step of determining whole gene copy numbers further comprises comparing the second coefficient of variation with a second threshold and calling a whole gene copy number variation for the BRCA2 gene if the second coefficient of variation is less than the second threshold.

Example 14 includes the subject matter of Example 5, and further specifies that the step of determining whole gene copy numbers further comprises multiplying a weight factor times the first mean to form a product.

Example 15 includes the subject matter of Example 14, and further specifies that the step of determining whole gene copy numbers further comprises determining if the product is less than the second mean.

Example 16 includes the subject matter of Example 5, and further specifies that the

step of determining whole gene copy numbers further comprises multiplying a weight factor times the second mean to form a product.

Example 17 includes the subject matter of Example 16, and further specifies that the step of determining whole gene copy numbers further comprises determining if the product is less than the first mean.

Example 18 includes the subject matter of Example 5, and further specifies that the step of determining whole gene copy numbers further comprises dividing the number of reads per amplicon for the sample ID amplicons by the total number of reads for the sample to form normalized read counts per amplicon for the sample ID amplicons.

Example 19 includes the subject matter of Example 18, and further specifies that the step of determining whole gene copy numbers further comprises calculating a third mean and a third standard deviation of the normalized read counts per amplicon for the sample ID (SID) amplicons.

Example 20 includes the subject matter of Example 19, and further specifies that the step of determining whole gene copy numbers further comprises applying a t-test based on the first and third means and the first and third standard deviations to determine a p-value.

Example 21 includes the subject matter of Example 20, and further specifies that the step of determining whole gene copy numbers further comprises comparing the p-value to a third threshold.

Example 22 includes the subject matter of Example 21, and further specifies that the step of determining whole gene copy numbers further comprises identifying a whole gene deletion or a whole gene amplification if the p-value is less than the third threshold.

Example 23 includes the subject matter of Example 19, and further specifies that the step of determining whole gene copy numbers further comprises multiplying a weight factor times the first mean to form a product.

Example 24 includes the subject matter of Example 23, and further specifies that the step of determining whole gene copy numbers further comprises comparing the product to the third mean.

Example 25 includes the subject matter of Example 19, and further specifies that the step of determining whole gene copy numbers further comprises applying a t-test based on the second and third means and the second and third standard deviations to determine a p-value.

Example 26 includes the subject matter of Example 25, and further specifies that the step of determining whole gene copy numbers further comprises comparing the p-value to a third threshold.

Example 27 includes the subject matter of Example 26, and further specifies that the step of determining whole gene copy numbers further comprises identifying a whole gene deletion or a whole gene amplification if the p-value is less than the third threshold.

Example 28 includes the subject matter of Example 19, and further specifies that the step of determining whole gene copy numbers further comprises multiplying a weight factor times the second mean to form a product.

Example 29 includes the subject matter of Example 28, and further specifies that the step of determining whole gene copy numbers further comprises comparing the product to the third mean.

Example 30 includes the subject matter of Example 19, and further specifies that the step of determining whole gene copy numbers further comprises: calculating a first coefficient of variation (CV) by dividing the first standard deviation by the first mean; calculating a second coefficient of variation (CV) by dividing the second standard deviation by the second mean; and calculating a third coefficient of variation (CV) by dividing the third standard deviation by the third mean.

Example 31 includes the subject matter of Example 30, and further specifies that the step of determining whole gene copy numbers further comprises: if the second mean is greater than the third mean and the first mean is not greater than the third mean, comparing the second CV to a CV threshold; comparing the third CV to a SID CV threshold; identifying a NOCALL for BRCA1 if the first CV is less than the CV threshold and the third CV is less than the SID CV threshold.

Example 32 includes the subject matter of Example 30, and further specifies that the step of determining whole gene copy numbers further comprises: if the first mean is greater than the third mean and the second mean is not greater than the third mean, comparing the first CV to a CV threshold; comparing the third CV to a SID CV threshold; identifying a NOCALL for BRCA2 if the first CV is less than the CV threshold and the third CV is less than the SID CV threshold.

Example 33 is a system for detecting gene level copy numbers for BRCA1 and BRCA2 genes, including: a machine-readable memory; and a processor configured to execute machine-readable instructions, which are configured to, when executed by the processor, cause the system to perform steps, comprising: receiving, at the processor, a plurality of sequence reads produced by amplifying a nucleic acid sample in a presence of a primer pool to produce a plurality of amplicons, the primer pool including a plurality of target-specific primers targeting regions of exons of the BRCA1 and BRCA2 genes and a plurality of sample ID regions, wherein the target-specific primers targeting the regions of exons produce overlapping amplicons that cover the exons of the BRCA1 and BRCA2 genes and the target-specific primers targeting the plurality of sample ID regions produce a plurality of sample ID amplicons, and sequencing the amplicons to generate the plurality of sequence reads; mapping the sequence reads to a reference genome, wherein the reference genome includes the BRCA1 and BRCA2 genes and the sample ID regions; determining a number of reads per amplicon for the amplicons associated with the exons of the BRCA1 gene, a number of reads per amplicon for the amplicons associated with the exons of the BRCA2 gene and a number of reads per amplicon for the sample ID amplicons associated with the sample ID regions; and determining whole gene copy numbers for the BRCA1 and BRCA2 genes based on the number of reads per amplicon for the amplicons associated with the exons of the BRCA1 gene, the number of reads per amplicon for the amplicons associated with the exons of the BRCA2 gene, and the number of reads per amplicon for the sample ID amplicons associated with the sample ID regions.

Example 34 includes the subject matter of Example 33, and further specifies that the sample ID amplicons are associated with different chromosomes than chromosome 17 containing the BRCA1 gene and chromosome 13 containing the BRCA2 gene.

Example 35 includes the subject matter of Example 33, and further specifies that the sample ID amplicons are distributed on different chromosomes unlinked to each other.

Example 36 includes the subject matter of Example 33, and further specifies that the step of determining whole gene copy numbers further comprises: dividing the number of reads per amplicon for the amplicons associated with the exons of the BRCA1 gene by a total number of reads for the sample to form normalized read counts per amplicon for the BRCA1 gene; and dividing the number of reads per amplicon for the amplicons associated with the exons of the BRCA2 gene by the total number of reads for the sample to form normalized read counts per amplicon for the BRCA2 gene.

Example 37 includes the subject matter of Example 36, and further specifies that the step of determining whole gene copy numbers further comprises: calculating a first mean and a first standard deviation of the normalized read counts per amplicon of the BRCA1 gene; and calculating a second mean and a second standard deviation of the normalized read counts per amplicon of the BRCA2 gene.

Example 38 includes the subject matter of Example 37, and further specifies that the step of determining whole gene copy numbers further comprises applying a t-test based on the first and second means and the first and second standard deviations to determine a p-value.

Example 39 includes the subject matter of Example 38, and further specifies that the step of determining whole gene copy numbers further comprises comparing the p-value to a first threshold.

Example 40 includes the subject matter of Example 39, and further specifies that the step of determining whole gene copy numbers further comprises identifying a whole gene deletion or a whole gene amplification if the p-value is less than the first threshold.

Example 41 includes the subject matter of Example 38, and further specifies that the step of determining whole gene copy numbers further comprises calculating a PHRED score based on the p-value.

Example 42 includes the subject matter of Example 41, and further specifies that the PHRED score is calculated by multiplying a logarithm of the p-value by (−10).

Example 43 includes the subject matter of Example 37, and further specifies that the step of determining whole gene copy numbers further comprises: calculating a first coefficient of variation (CV) by dividing the first standard deviation by the first mean; and calculating a second coefficient of variation (CV) by dividing the second standard deviation by the second mean.

Example 44 includes the subject matter of Example 43, and further specifies that the step of determining whole gene copy numbers further comprises comparing the first coefficient of variation with a second threshold and calling a whole gene copy number variation for the BRCA1 gene if the first coefficient of variation is less than the second threshold.

Example 45 includes the subject matter of Example 43, and further specifies that the step of determining whole gene copy numbers further comprises comparing the second coefficient of variation with a second threshold and calling a whole gene copy number variation for the BRCA2 gene if the second coefficient of variation is less than the second threshold.

Example 46 includes the subject matter of Example 37, and further specifies that the step of determining whole gene copy numbers further comprises multiplying a weight factor times the first mean to form a product.

Example 47 includes the subject matter of Example 46, and further specifies that the step of determining whole gene copy numbers further comprises determining if the product is less than the second mean.

Example 48 includes the subject matter of Example 37, and further specifies that the step of determining whole gene copy numbers further comprises multiplying a weight factor times the second mean to form a product.

Example 49 includes the subject matter of Example 48, and further specifies that the step of determining whole gene copy numbers further comprises determining if the product is less than the first mean.

Example 50 includes the subject matter of Example 37, and further specifies that the step of determining whole gene copy numbers further comprises dividing the number of reads per amplicon for the sample ID amplicons by the total number of reads for the sample to form normalized read counts per amplicon for the sample ID amplicons.

Example 51 includes the subject matter of Example 50, and further specifies that the step of determining whole gene copy numbers further comprises calculating a third mean and a third standard deviation of the normalized read counts per amplicon for the sample ID (SID) amplicons.

Example 52 includes the subject matter of Example 51, and further specifies that the step of determining whole gene copy numbers further comprises applying a t-test based on the first and third means and the first and third standard deviations to determine a p-value.

Example 53 includes the subject matter of Example 52, and further specifies that the step of determining whole gene copy numbers further comprises comparing the p-value to a third threshold.

Example 54 includes the subject matter of Example 53, and further specifies that the step of determining whole gene copy numbers further comprises identifying a whole gene deletion or a whole gene amplification if the p-value is less than the third threshold.

Example 55 includes the subject matter of Example 51, and further specifies that the step of determining whole gene copy numbers further comprises multiplying a weight factor times the first mean to form a product.

Example 56 includes the subject matter of Example 55, and further specifies that the step of determining whole gene copy numbers further comprises comparing the product to the third mean.

Example 57 includes the subject matter of Example 51, and further specifies that the step of determining whole gene copy numbers further comprises applying a t-test based on the second and third means and the second and third standard deviations to determine a p-value.

Example 58 includes the subject matter of Example 57, and further specifies that the step of determining whole gene copy numbers further comprises comparing the p-value to a third threshold.

Example 59 includes the subject matter of Example 58, and further specifies that the step of determining whole gene copy numbers further comprises identifying a whole gene deletion or a whole gene amplification if the p-value is less than the third threshold.

Example 60 includes the subject matter of Example 51, and further specifies that the step of determining whole gene copy numbers further comprises multiplying a weight factor times the second mean to form a product.

Example 61 includes the subject matter of Example 60, and further specifies that the step of determining whole gene copy numbers further comprises comparing the product to the third mean.

Example 62 includes the subject matter of Example 51, and further specifies that the step of determining whole gene copy numbers further comprises: calculating a first coefficient of variation (CV) by dividing the first standard deviation by the first mean; calculating a second coefficient of variation (CV) by dividing the second standard deviation by the second mean; and calculating a third coefficient of variation (CV) by dividing the third standard deviation by the third mean.

Example 63 includes the subject matter of Example 62, and further specifies that the step of determining whole gene copy numbers further comprises: if the second mean is greater than the third mean and the first mean is not greater than the third mean, comparing the second CV to a CV threshold; comparing the third CV to a SID CV threshold; identifying a NOCALL for BRCA1 if the first CV is less than the CV threshold and the third CV is less than the SID CV threshold.

Example 64 includes the subject matter of Example 62, and further specifies that the step of determining whole gene copy numbers further comprises: if the first mean is greater than the third mean and the second mean is not greater than the third mean, comparing the first CV to a CV threshold; comparing the third CV to a SID CV threshold; identifying a NOCALL for BRCA2 if the first CV is less than the CV threshold and the third CV is less than the SID CV threshold.

Example 65 is a non-transitory machine-readable storage medium comprising instructions which are configured to, when executed by a processor, cause the processor to perform a method for detecting gene level copy numbers for BRCA1 and BRCA2 genes, including: receiving, at the processor, a plurality of sequence reads produced by amplifying a nucleic acid sample in a presence of a primer pool to produce a plurality of amplicons, the primer pool including a plurality of target-specific primers targeting regions of exons of the BRCA1 and BRCA2 genes and a plurality of sample ID regions, wherein the target-specific primers targeting the regions of exons produce overlapping amplicons that cover the exons of the BRCA1 and BRCA2 genes and the target-specific primers targeting the plurality of sample ID regions produce a plurality of sample ID amplicons, and sequencing the amplicons to generate the plurality of sequence reads; mapping the sequence reads to a reference genome, wherein the reference genome includes the BRCA1 and BRCA2 genes and the sample ID regions; determining a number of reads per amplicon for the amplicons associated with the exons of the BRCA1 gene, a number of reads per amplicon for the amplicons associated with the exons of the BRCA2 gene and a number of reads per amplicon for the sample ID amplicons associated with the sample ID regions; and determining whole gene copy numbers for the BRCA1 and BRCA2 genes based on the number of reads per amplicon for the amplicons associated with the exons of the BRCA1 gene, the number of reads per amplicon for the amplicons associated with the exons of the BRCA2 gene, and the number of reads per amplicon for the sample ID amplicons associated with the sample ID regions.

Example 66 includes the subject matter of Example 65, and further specifies that the sample ID amplicons are associated with different chromosomes than chromosome 17containing the BRCA1 gene and chromosome 13 containing the BRCA2 gene.

Example 67 includes the subject matter of Example 65, and further specifies that the sample ID amplicons are distributed on different chromosomes unlinked to each other.

Example 68 includes the subject matter of Example 65, and further specifies that the step of determining whole gene copy numbers further comprises: dividing the number of reads per amplicon for the amplicons associated with the exons of the BRCA1 gene by a total number of reads for the sample to form normalized read counts per amplicon for the BRCA1 gene; and dividing the number of reads per amplicon for the amplicons associated with the exons of the BRCA2 gene by the total number of reads for the sample to form normalized read counts per amplicon for the BRCA2 gene.

Example 69 includes the subject matter of Example 68, and further specifies that the step of determining whole gene copy numbers further comprises: calculating a first mean and a first standard deviation of the normalized read counts per amplicon of the BRCA1 gene; and calculating a second mean and a second standard deviation of the normalized read counts per amplicon of the BRCA2 gene.

Example 70 includes the subject matter of Example 69, and further specifies that the step of determining whole gene copy numbers further comprises applying a t-test based on the first and second means and the first and second standard deviations to determine a p-value.

Example 71 includes the subject matter of Example 70, and further specifies that the step of determining whole gene copy numbers further comprises comparing the p-value to a first threshold.

Example 72 includes the subject matter of Example 71, and further specifies that the step of determining whole gene copy numbers further comprises identifying a whole gene deletion or a whole gene amplification if the p-value is less than the first threshold.

Example 73 includes the subject matter of Example 70, and further specifies that the step of determining whole gene copy numbers further comprises calculating a PHRED score based on the p-value.

Example 74 includes the subject matter of Example 73, and further specifies that the PHRED score is calculated by multiplying a logarithm of the p-value by (−10).

Example 75 includes the subject matter of Example 69, and further specifies that the step of determining whole gene copy numbers further comprises: calculating a first coefficient of variation (CV) by dividing the first standard deviation by the first mean; and calculating a second coefficient of variation (CV) by dividing the second standard deviation by the second mean.

Example 76 includes the subject matter of Example 75, and further specifies that the step of determining whole gene copy numbers further comprises comparing the first coefficient of variation with a second threshold and calling a whole gene copy number variation for the BRCA1 gene if the first coefficient of variation is less than the second threshold.

Example 77 includes the subject matter of Example 75, and further specifies that the step of determining whole gene copy numbers further comprises comparing the second coefficient of variation with a second threshold and calling a whole gene copy number variation for the BRCA2 gene if the second coefficient of variation is less than the second threshold.

Example 78 includes the subject matter of Example 69, and further specifies that the step of determining whole gene copy numbers further comprises multiplying a weight factor times the first mean to form a product.

Example 79 includes the subject matter of Example 78, and further specifies that the step of determining whole gene copy numbers further comprises determining if the product is less than the second mean.

Example 80 includes the subject matter of Example 69, and further specifies that the step of determining whole gene copy numbers further comprises multiplying a weight factor times the second mean to form a product.

Example 81 includes the subject matter of Example 80, and further specifies that the step of determining whole gene copy numbers further comprises determining if the product is less than the first mean.

Example 82 includes the subject matter of Example 69, and further specifies that the step of determining whole gene copy numbers further comprises dividing the number of reads per amplicon for the sample ID amplicons by the total number of reads for the sample to form normalized read counts per amplicon for the sample ID amplicons.

Example 83 includes the subject matter of Example 82, and further specifies that the step of determining whole gene copy numbers further comprises calculating a third mean and a third standard deviation of the normalized read counts per amplicon for the sample ID (SID) amplicons.

Example 84 includes the subject matter of Example 83, and further specifies that the step of determining whole gene copy numbers further comprises applying a t-test based on the first and third means and the first and third standard deviations to determine a p-value.

Example 85 includes the subject matter of Example 84, and further specifies that the step of determining whole gene copy numbers further comprises comparing the p-value to a third threshold.

Example 86 includes the subject matter of Example 85, and further specifies that the step of determining whole gene copy numbers further comprises identifying a whole gene deletion or a whole gene amplification if the p-value is less than the third threshold.

Example 87 includes the subject matter of Example 83, and further specifies that the step of determining whole gene copy numbers further comprises multiplying a weight factor times the first mean to form a product.

Example 88 includes the subject matter of Example 87, and further specifies that the step of determining whole gene copy numbers further comprises comparing the product to the third mean.

Example 89 includes the subject matter of Example 83, and further specifies that the step of determining whole gene copy numbers further comprises applying a t-test based on the second and third means and the second and third standard deviations to determine a p-value.

Example 90 includes the subject matter of Example 89, and further specifies that the step of determining whole gene copy numbers further comprises comparing the p-value to a third threshold.

Example 91 includes the subject matter of Example 90, and further specifies that the step of determining whole gene copy numbers further comprises identifying a whole gene deletion or a whole gene amplification if the p-value is less than the third threshold.

Example 92 includes the subject matter of Example 83, and further specifies that the step of determining whole gene copy numbers further comprises multiplying a weight factor times the second mean to form a product.

Example 93 includes the subject matter of Example 92, and further specifies that the step of determining whole gene copy numbers further comprises comparing the product to the third mean.

Example 94 includes the subject matter of Example 83, and further specifies that the step of determining whole gene copy numbers further comprises: calculating a first coefficient of variation (CV) by dividing the first standard deviation by the first mean; calculating a second coefficient of variation (CV) by dividing the second standard deviation by the second mean; and calculating a third coefficient of variation (CV) by dividing the third standard deviation by the third mean.

Example 95 includes the subject matter of Example 93, and further specifies that the step of determining whole gene copy numbers further comprises: if the second mean is greater than the third mean and the first mean is not greater than the third mean, comparing the second CV to a CV threshold; comparing the third CV to a SID CV threshold; identifying a NOCALL for BRCA1 if the first CV is less than the CV threshold and the third CV is less than the SID CV threshold.

Example 96 includes the subject matter of Example 93, and further specifies that the step of determining whole gene copy numbers further comprises: if the first mean is greater than the third mean and the second mean is not greater than the third mean, comparing the first CV to a CV threshold; comparing the third CV to a SID CV threshold; identifying a NOCALL for BRCA2 if the first CV is less than the CV threshold and the third CV is less than the SID CV threshold.

What is claimed is:

1. A method for detecting gene level copy numbers for BRCA1 and BRCA2 genes, comprising:
amplifying a nucleic acid sample in a presence of a primer pool to produce a plurality of amplicons, the primer pool including a plurality of target-specific primers targeting regions of exons of the BRCA1 and BRCA2 genes and a plurality of sample ID regions, wherein the target-specific primers targeting the regions of exons produce overlapping amplicons that cover the exons of the BRCA1 and BRCA2 genes and the target-specific primers targeting the plurality of sample ID regions produce a plurality of sample ID amplicons;
sequencing the amplicons to generate a plurality of sequence reads;
mapping the sequence reads to a reference genome, wherein the reference genome includes the BRCA1 and BRCA2 genes and the sample ID regions;
determining a number of reads per amplicon for the amplicons associated with the exons of the BRCA1 gene, a number of reads per amplicon for the amplicons associated with the exons of the BRCA2 gene and a number of reads per amplicon for the sample ID amplicons associated with the sample ID regions; and
determining whole gene copy numbers for the BRCA1 and BRCA2 genes based on the number of reads per amplicon for the amplicons associated with the exons of the BRCA1 gene, the number of reads per amplicon for the amplicons associated with the exons of the BRCA2 gene, and the number of reads per amplicon for the sample ID amplicons associated with the sample ID regions.

2. The method of claim 1, wherein the step of determining whole gene copy numbers further comprises:
dividing the number of reads per amplicon for the amplicons associated with the exons of the BRCA1 gene by a total number of reads for the sample to form normalized read counts per amplicon for the BRCA1 gene; and
dividing the number of reads per amplicon for the amplicons associated with the exons of the BRCA2 gene by the total number of reads for the sample to form normalized read counts per amplicon for the BRCA2 gene.

3. The method of claim 2, wherein the step of determining whole gene copy numbers further comprises:
calculating a first mean and a first standard deviation of the normalized read counts per amplicon of the BRCA1 gene; and
calculating a second mean and a second standard deviation of the normalized read counts per amplicon of the BRCA2 gene.

4. The method of claim 3, wherein the step of determining whole gene copy numbers further comprises applying a t-test based on the first and second means and the first and second standard deviations to determine a p-value.

5. The method of claim 4, wherein the step of determining whole gene copy numbers further comprises comparing the p-value to a first threshold.

6. The method of claim 5, wherein the step of determining whole gene copy numbers further comprises identifying a whole gene deletion or a whole gene amplification if the p-value is less than the first threshold.

7. The method of claim 3, wherein the step of determining whole gene copy numbers further comprises:
calculating a first coefficient of variation (CV) by dividing the first standard deviation by the first mean; and
calculating a second coefficient of variation (CV) by dividing the second standard deviation by the second mean.

8. The method of claim 7, wherein the step of determining whole gene copy numbers further comprises comparing the first coefficient of variation with a second threshold and calling a whole gene copy number variation for the BRCA1 gene if the first coefficient of variation is less than the second threshold.

9. The method of claim 7, wherein the step of determining whole gene copy numbers further comprises comparing the second coefficient of variation with a second threshold and calling a whole gene copy number variation for the BRCA2 gene if the second coefficient of variation is less than the second threshold.

10. The method of claim 3, wherein the step of determining whole gene copy numbers further comprises dividing the number of reads per amplicon for the sample ID amplicons by the total number of reads for the sample to form normalized read counts per amplicon for the sample ID amplicons.

11. The method of claim 10, wherein the step of determining whole gene copy numbers further comprises calculating a third mean and a third standard deviation of the normalized read counts per amplicon for the sample ID (SID) amplicons.

12. The method of claim 11, wherein the step of determining whole gene copy numbers further comprises applying a t-test based on the first and third means and the first and third standard deviations to determine a p-value.

13. The method of claim 12, wherein the step of determining whole gene copy numbers further comprises comparing the p-value to a third threshold.

14. The method of claim 13, wherein the step of determining whole gene copy numbers further comprises identifying a whole gene deletion or a whole gene amplification if the p-value is less than the third threshold.

15. The method of claim 11, wherein the step of determining whole gene copy numbers further comprises applying a t-test based on the second and third means and the second and third standard deviations to determine a p-value.

16. The method of claim 15, wherein the step of determining whole gene copy numbers further comprises comparing the p-value to a third threshold.

17. The method of claim 16, wherein the step of determining whole gene copy numbers further comprises identifying a whole gene deletion or a whole gene amplification if the p-value is less than the third threshold.

18. The method of claim 11, wherein the step of determining whole gene copy numbers further comprises:
calculating a first coefficient of variation (CV) by dividing the first standard deviation by the first mean;
calculating a second coefficient of variation (CV) by dividing the second standard deviation by the second mean; and
calculating a third coefficient of variation (CV) by dividing the third standard deviation by the third mean.

19. The method of claim 18, wherein the step of determining whole gene copy numbers further comprises:
if the second mean is greater than the third mean and the first mean is not greater than the third mean,
comparing the second CV to a CV threshold;
comparing the third CV to a SID CV threshold; and
identifying a NOCALL for BRCA1 if the first CV is less than the CV threshold and the third CV is less than the SID CV threshold.

20. The method of claim 18, wherein the step of determining whole gene copy numbers further comprises:
if the first mean is greater than the third mean and the second mean is not greater than the third mean,
comparing the first CV to a CV threshold;
comparing the third CV to a SID CV threshold; and
identifying a NOCALL for BRCA2 if the first CV is less than the CV threshold and the third CV is less than the SID CV threshold.
